(19) United States
(12) Patent Application Publication
(10) Pub. No.: US 2006/0275249 A1
(43) Pub. Date: Dec. 7, 2006

(54) Co-therapy for diabetic conditions

(76) Inventor: Michael R. Jones, New York, NY (US)

Correspondence Address: Karen M. Whitney;DIEHL SERVILLA LLC, Suite 110, 77 Brant Ave., Clark, NJ, 07066, US

(21) Appl. No.: 11/446,054

(22) Filed: Jun. 2, 2006

Related U.S. Application Data
(60) Provisional application No. 60/687,206, filed on Jun. 3, 2005.

(51) Int. Cl. A61K 31/785; A61K 31/155

(52) U.S. Cl. 424/78.14; 514/635

(57) ABSTRACT

Methods of treating diseases diabetes are disclosed. Methods of modulating elevated fructosamine levels, elevated HbA1c levels, impaired glucose tolerance, and impaired fasting glucose are also disclosed. In some embodiments, methods include co-administration of a biguanide and a bile acid sequestrant. Drug products including a biguanide and bile acid sequestrants in combination are also disclosed.

### CROSS-REFERENCE TO RELATED APPLICATION

This application claims the benefit of the filing date under the provisions of 35 U.S.C. § 119(e) of provisional patent application Ser. No. 60/687,206, filed Jun. 3, 2005, which is incorporated herein by reference in its entirety.

### BACKGROUND OF THE INVENTION

The present invention generally relates to diabetic conditions and drug products for treatment of these conditions.

At least about 16 million Americans have type 2 diabetes. Individuals afflicted with type 2 and type 1 diabetes have elevated blood sugar levels due to problems with either the amount of or action of insulin, which regulates the body's handling of glucose. In type 1 diabetes, the pancreas is unable to respond normally to blood sugar levels by secreting insulin. In type 2 diabetes, the more common form, the liver and peripheral tissues may be less responsive to insulin. In later stages of type 2 diabetes, the pancreas may also secrete inadequate amounts of insulin for proper blood sugar control. Diabetic individuals who control blood glucose levels can substantially reduce the risk of developing vascular complications of diabetes, including, but not limited, to diabetic retinopathy (a condition which leads to blindness), diabetic nephropathy, diabetic neuropathy, and atherosclerosis.

The American Diabetes Association has recommended that patients with type 2 diabetes be treated to a goal of glycosylated hemoglobin A (HbA1c) of <7%, the level at which clinical trials have demonstrated fewer long-term microvascular complications. From the Third National Health and Nutrition Examination Survey data, it appears that only about 40% of patients with type 2 diabetes achieve this goal.

Taking care of patients with diabetes mellitus and its complications is estimated to cost more than $132 billion each year. Much of the personal and economic burden related to the care of diabetic patients stems from inadequate glycemic control. Studies have demonstrated that glycemic control in the majority of patients with type 2 diabetes is inadequate. The position statement of the ADA recommends that all patients with type 2 diabetes be treated with diet, exercise, and when necessary, with medication to bring their HbA1c levels to below a threshold of 7%. An epidemiological analysis of the UK Prospective Diabetes Study data demonstrated an approximate 14% reduction in all-cause mortality and myocardial infarction for every 1% reduction in HbA1c. Furthermore, it is estimated that there is a 15%-30% reduction in the risk of microvascular complications for each 1% reduction in HbA1c.

Current methods of controlling blood glucose concentration include insulin injections or oral administration of sulfonylureas (e.g., glyburide), biguanide drugs (e.g., metformin), alpha-glucosidase inhibitors (e.g., acarbose), or thiazolidinedione. Metformin improves glucose tolerance in diabetic patients by lowering both basal and postprandial plasma glucose. Metformin hydrochloride is currently sold under the trademark Glucophage® in tablet form by Bristol-Myers Squibb Co. Glucophage® tablets are provided in dosage amounts containing 500, 850 or 1000 mg metformin hydrochloride. Glucophage® dosages are not fixed, and dosages are typically individualized on the basis of both effectiveness and tolerance, while not exceeding the maximum recommended dose of 2550 mg per day. Patients on metformin therapy may also be prescribed another drug.

Metformin has been widely prescribed for lowering blood glucose in patients with diabetes. Patients prescribed metformin must receive twice-daily (b.i.d.) or three-times-a-day (t.i.d.) dosing. Adverse events associated with metformin use are often gastrointestinal in nature (e.g., anorexia, nausea, vomiting and occasionally diarrhea, etc.). It would be desirable to improve metformin therapy, and in particular, it would be desirable to minimize undesirable adverse events.

### SUMMARY OF THE INVENTION

One aspect of the present invention pertains to methods for the treatment of diabetes and diabetic conditions and modulating elevated fructosamine, HbA1c levels, impaired glucose tolerance or impaired fasting glucose. According to one embodiment, treatment is effected by co-administering to a patient in need thereof a therapeutically effective amount of a biguanide agent and a therapeutically effective amount of a bile acid sequestrant. Specific embodiments involve the administration of therapeutically effective amounts of metformin and colesevelam. Another embodiment of the invention relates to a method of modulating blood glucose levels in a mammal by co-administering a biguanide such as metformin and a bile acid sequestrant such as colesevelam.

A second aspect of the present invention pertains to a drug product incorporating a biguanide agent and a bile acid sequestrant. The drug product can be provided in a single dosage form incorporating a biguanide agent and a bile acid sequestrant. Alternatively, the biguanide and the bile acid sequestrant can be separated in a single container or package with instructions for co-administration.

### DETAILED DESCRIPTION

It is to be appreciated that the various method steps described herein include approximations of dosage amounts and may be varied. Before describing several exemplary embodiments of the invention, it is to be understood that the invention is not limited to the details set forth in the following description. The invention is capable of other embodiments and of being practiced or carried out in various ways.

In overview, one aspect of the invention pertains to methods for the treatment of diabetes. Treatment may be effected by the co-administration to a patient or subject a biguanide agent and a bile acid sequestrant. As used herein, co-administration means administering a dosage of a biguanide agent within twelve hours of administration of a bile acid sequestrant to the same patient or subject. In one embodiment, a method of modulating blood glucose levels in a human in need thereof by co-administering to a patient or a subject a biguanide such as metformin and a bile acid sequestrant such as colesevelam is provided.

In other embodiments, methods of modulating elevated fructosamine and/or HbA1c levels are provided, for example, in patients afflicted with type 2 diabetes or non-insulin dependent (NIDDM) diabetes mellitus.

In further embodiments, methods are provided for modulating impaired glucose tolerance or impaired fasting glucose. Impaired glucose tolerance is generally defined as two-hour glucose levels of 140 to 199 mg per dL (7.8 to 11.0 mmol per L) on the 75 g glucose tolerance test. Impaired fasting glucose is generally defined as glucose levels of 100 to 125 mg per dL (5.6 to 6.9 mmol per L) in fasting patients. Patients with impaired glucose tolerance or impaired fasting glucose have a significant risk of developing diabetes and are an important group for preventing diabetes. In the present specification, the meaning of terms “active agent”, “active compound” or in some cases “compound” should be understood as equivalent.

Also provided by this invention is a method for modulating blood glucose levels in a mammal in need thereof. Modulating blood glucose levels as used herein is understood to indicate maintaining glucose levels within clinically normal ranges or lowering elevated blood glucose levels to a more clinically desirable level or range.

According to one or more embodiments, the methods comprise administering to said mammal co-therapy of (1) a biguanide selected from the group consisting of metformin and pharmaceutically acceptable salts thereof, and (2) a bile acid sequestrant selected from the group consisting of colesevelam (commercially known as Welchol®, cholestyramine (commercially known as Questran®, Cholybar®), colestipol (commercially known as Colestid®) and pharmacueutically acceptable salts thereof of each of these bile acid sequestrants, the biguanide and the bile acid sequestrant being administered in therapeutically effective amounts to treat said condition.

As used herein, a pharmaceutically or therapeutically effective amount is understood to be at least a minimal amount which provides a medical improvement in the symptoms of the specific malady or disorder experienced by the mammal in question. Preferably, the recipient will experience a reduction, inhibition or removal of the biological basis for the malady in question.

A presently preferred salt of metformin is metformin hydrochloride, although the present invention is not limited to a particular salt. Metformin hydrochloride useful in the methods and combinations is commercially available in 500 mg, 850 mg and 1000 mg tablets under the Glucophage® trademark from Bristol Meyers Squibb. Metformin hydrochloride may be administered in humans at an initial daily dose of from 500 mg to about 1000 mg and increased, as needed, to a maximum daily dosage of 2550 mg. A potential limiting factor in the use of metformin is its tendency to cause gastrointestinal disorders, primarily diarrhea. This side effect has been studied in a series of studies in diabetic patients, in rats, and in cultured human intestinal cells. These studies consistently found that metformin causes a state of malabsorption of bile acid salts from the intestine. The appearance of excess unabsorbed bile salts in the intestine exerts an osmotic effect that is known to cause diarrhea. The severity of diarrhea with metformin usage may limit the dose that can be given and, hence, the efficacy of the treatment.

A presently preferred bile acid sequestrant includes colesevelam hydrochloride sold under the trademark Welchol® by Sankyo Pharma Inc. Generally the daily dosage colesevelam hydrochloride is between about 1.5 grams and 3.75 grams and usually does not exceed 3.75 grams per day. One typical side effect of treatment with colesevelam HCl is constipation. This is partly or wholly due to the binding of bile acid salts in the intestine. The binding of bile acid salts to the polymer colesevelam HCl in the intestine results in a lower osmotic effect favoring constipation rather than diarrhea. In a patient receiving metformin, therefore, concomitant treatment with colesevelam HCl may mitigate the diarrhea and allow greater and more effective doses of metformin to be used.

Other dosage forms are within the scope of the present invention. The dosages may be varied depending upon the requirements of the patient, the severity of the condition being treated and the compound being employed. Determination of the proper dosage for a particular situation is within the skill of the art. In one embodiment, generally, treatment is initiated with smaller dosages which are less than the optimum dose of the compound. Thereafter, the dosage is increased by small increments until the optimum effect under the circumstance is reached.

It is understood that the dosage, regimen and mode of administration of these compounds will vary according to the condition and the individual being treated and will be subject to the judgment of the medical practitioner involved. It is preferred that the administration of one or more of the compounds herein begin at a low dose and be increased until the desired effects are achieved. It is also preferred that the recipient also utilize art recognized lifestyle patterns for reducing the incidence of the maladies described herein. These include maintenance of an appropriate diet and exercise regimen, as recommended by a medical practitioner familiar with the physical condition of the recipient.

While the biguanide and the bile acid sequestrant can be administered at different times, they also can be administered at the same time. When the biguanide and the bile acid sequestrant are given substantially simultaneously, they may be given by a single fixed combination dosage form or by different dosage forms, whichever is convenient.

When given in different dosage forms, it is irrelevant whether the route of administration is the same for each agent or different for each agent. Any route of administration known for the individual agents is acceptable for the practice of the present invention. The agents can be given in a fixed combination, or at least substantially simultaneously, i.e. within about 1 hour of each other. Also, the most suitable dosage form is an oral dosage form, where oral administration is a clinically suitable route. However, the biguanide can be administered at times different from the administration of the bile acid sequestrant, and the invention benefits may still be realized. When administered at different times, it is believed that the biguanide and the bile acid sequestrant should be given within about twelve hours of each other, preferably within about four hours of each other, and more preferably within about two hours of each other. Of course, these time periods can be adjusted if the dosage form is one which will “administer” the agents for extended periods.

Dosages of the two agents include all dosages at which the agents are used individually as discussed above. The proper dosage for each agent can be obtained from any convenient reference such as the Physician's Desk Reference (PDR) or the label for each agent. Modified dosage ranges for mammals of varying sizes and stages of development will be apparent to those of ordinary skill.

It may be desirable if the biguanide and bile acid sequestrant are incorporated into a single dosage form to keep the agents physically separated. This may be accomplished in any of the myriad ways known in the art, such as bi-layered tablets, coated pellets of one agent incorporated into a tablet of the other, separately coated pellets of each agent in a capsule or tablet, coated pellets of one agent in capsule together with powder of the other agent, each agent microencapsulated separately and then blended together for use in a tablet or capsule, use of a dual or multiple compartment transdermal device, etc.

The biguanide and bile acid sequestrants of the present invention can be prepared and administered in a wide variety of oral dosage forms.

According to one or more embodiments of the invention, the preparation of pharmaceutical compositions can involve the use of pharmaceutically acceptable carriers, which can be either solid or liquid. Solid form preparations include powders, tablets, pills, capsules, sachets, suppositories, and dispersible granules. A solid carrier can be one or more substances which may also act as diluents, flavoring agents, solubilizers, lubricants, suspending agents, binders, preservatives, tablet disintegrating agents, an encapsulating material, or drug delivery agents, such as liposomal preparations.

One or more embodiments of the invention include solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

In embodiments including powders, the carrier typically is a finely divided solid which is in a mixture with the finely divided active component. In embodiments including tablets, the active component is mixed with the carrier having the necessary binding properties in suitable proportions and compacted in the shape and size desired.

Suitable carriers include, but are not limited to, magnesium carbonate, magnesium stearate, talc, sugar, lactose, pectin, dextrin, starch, gelatin, tragacanth, methyl cellulose, sodium carboxymethyl cellulose, a low melting wax, cocoa butter, and the like. The term “preparation” is intended to include the formulation of the active compound with encapsulating material as a carrier providing a capsule in which the active component, with or without other carriers, is surrounded by a carrier, which is thus in association with it. Similarly, cachets and lozenges are included. Tablets, powders, capsules, pills, cachets, and lozenges can be used as solid dosage forms suitable for oral administration.

Liquid form preparations include solutions, suspensions, and emulsions, for example, water or water propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizing, and thickening agents as desired. Aqueous suspensions suitable for oral use can be made by dispersing the finely divided active component in water with viscous material, such as natural or synthetic gums, resins, methyl cellulose, sodium carboxymethyl cellulose, and other well-known suspending agents.

The pharmaceutical preparations are preferably in unit dosage form. In such form, the preparations are subdivided into unit doses containing appropriate quantities of the active component. The unit dosage form can be a packaged preparation, the package containing discrete quantities of preparation, such as packeted tablets, capsules, and powders in vials or ampules. Also, the unit dosage form can be a capsule, tablet, cachet, or lozenge itself, or it can be the appropriate number of any of these in packaged form.

Exemplary embodiments of the invention will be further described for illustrative purposes with reference to the following non-limiting examples.

### EXAMPLE 1

Administration of Colesevelam to Diabetic Patients Taking Metformin Alone or With Another Drug

A prospective, randomized, double-blind, placebo-controlled, parallel group study, consisting of a 5-week, placebo run-in period (i.e., 1 week of screening and then 4 weeks of placebo treatment) followed by a 12-week active treatment period was conducted. Eligible patients were randomized to either WelChol® (3.75 g/day in 6 tablets/day) or placebo (6 tablets/day). Enrollment was limited to patients with type 2 diabetes who were receiving a stable dose of treatment with a sulfonylurea, metformin, or the combination of metformin and a sulfonylurea, and whose glucose was not adequately controlled at a third visit (HbA1c 7.0% to 10.0%, inclusive). Patients who met the initial entry criteria were re-evaluated 4 weeks later to confirm the stability of their HbA1c measurement (i.e., did not differ from the initial screening value by more than 0.5%). Patients who met this criterion and the other entry criteria were then randomized to receive either WelChol® (3.75 g/day in 6 tablets/day) or placebo (6 tablets/day) for 12 weeks.

As noted above, prior to entering the study, patients were receiving sulfonylurea, metformin, or the combination of metformin and a sulfonylurea. The dose of antidiabetic medication must have been stable for 90 days prior to Visit 1 (Week −5). All other antidiabetic agents were to be discontinued for at least 90 days prior to Visit 1 (Week −5). The use of any other investigational drug was prohibited. A total of 27 (41.5%) patients took a sulfonylurea alone, 9 (13.8%) patients took metformin, and 29 (44.6%) patients took the combination.

The primary efficacy parameter was the change in HbA1c from Week 0 (baseline) to Week 12. The secondary efficacy parameters included the change in HbA1c from baseline to Weeks 4 and 8; the change in fructosamine from baseline to Weeks 4, 8, and 12; the change in protein-bound glucose from baseline to Weeks 4, 8, and 12; the change in fasting plasma glucose (FPG) from baseline to Weeks 4, 8, and 12; the change in meal glucose response from baseline to Weeks 1 and 12; the change in preprandial and postprandial glucose from baseline to Weeks 1 and 12; the change in free fatty acids from baseline to Weeks 4, 8, and 12; the change in insulin from baseline to Weeks 4, 8, and 12; and the change in homeostasis model assessment (HOMA) index from baseline to Weeks 4, 8, and 12. Percent changes in lipid parameters and changes in lipid subfractions were also evaluated.

The primary null hypothesis was that there was no difference between the treatment groups in the primary efficacy parameter, change in HbA1c from baseline to Week 12. When normality of the data was not violated, a mixed effect analysis of covariance (ANCOVA) model with treatment group as a fixed effect, center as a random effect, and the corresponding baseline value as a covariate was used. Least-squares (LS) mean, standard error, corresponding 95% confidence interval, and p-value were calculated for the treatment difference. The approach used for analysis of the primary efficacy parameter was also applied to analysis of the secondary diabetic efficacy parameters. A mixed effect analysis of variance model with treatment group (fixed effect) and center (random effect) as factors was used to analyze the percent change in lipid parameters and change in lipid subfractions from baseline to Week 12.

Following 12 weeks of treatment, HbA1c was reduced by 0.3% in the WelChol® group and increased by 0.2% in the placebo group. The LS mean treatment difference was statistically significant (−0.5%; p=0.007). A subgroup analysis demonstrated that for patients with HbA1c ≧8.0%, the mean change in HbA1c from baseline to Week 12 was −0.7% for the WelChol group and 0.2% for the placebo group; the LS mean treatment difference was statistically significant (−1.0%; p=0.002). A post-hoc subgroup analysis showed that for patients with HbA1c ≦7.5%, the LS mean change in HbA1c from baseline to Week 12 was −0.5% for the WelChol® group and 0.2% for the placebo group; the LS mean treatment differences was statistically significant (−0.8%; p=0.001).

Following 12 weeks of treatment, fructosamine was reduced by 10.9 μmol/L in the WelChol® group and increased by 11.7 μmol/L in the placebo group, yielding a statistically significant treatment difference (−29.0 μmol/L; p=0.011). Throughout the study, FPG was reduced to a greater extent in the WelChol® group compared to the placebo group. Although the LS mean treatment difference in FPG was statistically significant at Week 4 (−23.3 mg/dL; p=0.016) and Week 8 (−18.3 mg/dL; p=0.011), the difference between the WelChol® and the placebo treatment groups was not statistically significant at Week 12 (−14.0 mg/dL; p=0.118).

At Week 12, postprandial glucose was reduced by 17.8 mg/dL in the WelChol® group compared to a 2.7 mg/dL increase in the placebo group; the LS mean difference between the treatment groups was statistically significant (−31.5 mg/dL; p=0.026). There were no statistically significant treatment differences with regard to changes from baseline in protein-bound glucose, meal glucose response, free fatty acids, insulin, or HOMA index.

Treatment with WelChol® over a 12-week period resulted in statistically significant percent reductions in low-density lipoprotein cholesterol (−11.7%; p=0.007), total cholesterol (−7.3%; p=0.019), and apolipoprotein B (−11.8%; p=0.003) compared to placebo, and a statistically significant reduction in low-density lipoprotein particle concentration (−209.6 nmol/L; p=0.037) compared to placebo. There were no statistically significant treatment differences with regard to any other lipid parameters.

Treatment with WelChol® for 12 weeks resulted in statistically significant reductions in HbA1c, fructosamine, and postprandial glucose compared to treatment with placebo. These results demonstrate that a bile acid sequestrant such as WelChol® may be a useful agent for improving glycemic control in patients with type 2 diabetes mellitus who are on metformin therapy or metformin therapy in combination with another agent, such as a sulfonylurea. According to the present invention other bile acid sequestrants may be administered to patients receiving metformin therapy alone or metformin therapy combined with another drug. Addition of WelChol® to treatment did not result in any new, unexpected safety or tolerability issues.

The treatment difference in HbA1c of 0.5% reduction in the WelChol® group compared to the placebo group is both highly statistically significant (p=0.007) and clinically meaningful. The importance of the reduction in HbA1c is further demonstrated by a treatment difference in HbA1c levels of −0.8% and −1.0% in the subgroups of patients with a baseline HbA1c ≧7.5% (p=0.001) and ≧8.0% (p=0.002), respectively. The results of these subgroup analyses suggest that WelChol®, when used as an agent to improve glycemic control, may be more useful in patients who are most in need of additional drug support (i.e., those individuals with higher HbA1c levels and already on metformin therapy alone or in combination with another drug).

In addition to the beneficial effects observed in the diabetic parameters, WelChol® also reduced LDL-C, LDL particle concentration, Total-C, and apo B levels. Compared to placebo, treatment with WelChol® over a 12-week period resulted in statistically significant mean percent reductions in LDL-C (−11.7%; p=0.007), Total-C (−7.3%; p=0.019), and apo B (−11.8%; p=0.003), and a statistically significant mean reduction in LDL particle concentration (−209.6 nmol/L; p=0.037). The improvement in both glycemic and lipid parameters contributes to a reduction in the global risk for coronary heart disease in these high-risk patients with diabetes.

All publications cited in the specification, both patent publications and non-patent publications, are indicative of the level of skill of those skilled in the art to which this invention pertains. All these publications are herein fully incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed:

1. A method for treating diabetes in a human, the
method comprising administering to a human in need thereof a pharmaceutically effective amount of a biguanide agent and a pharmaceutically effective amount of a bile acid sequestrant.

2. The method of claim 1, wherein the biguanide agent and the bile acid sequestrant are administered simultaneously.

3. The method of claim 2, wherein the bile acid sequestrant and the biguanide agent are administered in a single dosage form.

4. The method of claim 1, wherein the bile acid sequestrant and the biguanide agent are administered separately.

5. The method of claim 4, wherein the bile acid sequestrant and the biguanide agent are administered within one hour of each other.

6. The method of claim 4, wherein the bile acid sequestrant and the biguanide agent are administered within twelve hours of each other.

7. The method of claim 1, wherein the biguanide agent comprises metformin or a pharmaceutically acceptable salt thereof.

8. The method of claim 7, wherein the bile acid sequestrant comprises colesevelam or a pharmaceutically acceptable salt thereof.

9. The method of claim 8, wherein the salt form of colesevelam comprises colesevelam HCl and the salt form of metformin comprises metformin HCl.

10. A method for modulating a condition in a subject,
the condition selected from the group consisting of elevated fructosamine levels, elevated HbA1c levels, impaired glucose tolerance, and impaired fasting glucose comprising co-administering to a subject a bile acid sequestrant, in free or pharmaceutically acceptable salt form; and a biguanide in free or pharmaceutically acceptable salt form, the biguanide and the bile acid sequestrant being administered in therapeutically effective amounts to treat said condition.

11. The method of claim 10, wherein the biguanide is selected from the group consisting of metformin and pharmaceutically acceptable salts thereof, and the bile acid sequestrant is selected from the group consisting of colesevelam, cholestyramine, colestipid, and pharmacueutically acceptable salts thereof.

12. The method of claim 10, wherein the biguanide comprises metformin or a pharmaceutically acceptable salt thereof and the bile acid sequestrant comprises colesevelam or a pharmaceutically acceptable salt thereof.

13. A drug product comprising a unit dosage of
metformin, in free or pharmaceutically acceptable salt form, in an amount corresponding to from about 500 mg to about 1000 mg of metformin; and at least about 3.75 g of colesevelam, in free or pharmaceutically acceptable salt form.

14. The drug product of claim 13, wherein the colesevelam and the metformin are physically separated from each other.
